(19) United States
(12) Patent Application Publication
(10) Pub. No.: US 2024/0383834 A1
(43) Pub. Date: Nov. 21, 2024

(54) METHOD FOR IMPROVED CONTROL OF THE ISOMER RATIOS IN HYDROFORMYLATIONS

(71) Applicant: OQ CHEMICALS GMBH, Monheim am Rhein (DE)

(72) Inventors: Christoph BALZAREK, Krefeld (DE); Lars HÜTTERMANN, Gelsenkirchen (DE); Leif JOHNEN, Dorsten (DE); Gregor MEIER, Duisburg (DE); Marcel MUSENBROCK, Düsseldorf (DE); Julian OELJEKLAUS, Essen (DE); Alexander STEFAN, Düsseldorf (DE); Dennis VOGELSANG, Dülmen (DE)

(73) Assignee: OQ CHEMICALS GMBH, Monheim am Rhein (DE)

(21) Appl. No.: 18/688,769

(22) PCT Filed: Sep. 20, 2022
(86) PCT No.: PCT/EP2022/076089

§ 371 Date: Mar. 3, 2024

(30) Foreign Application Priority Data
Sep. 24, 2021 (EP) 21198950.4

(51) Int. Cl. C07C 45/50; B01J 31/24

(52) U.S. Cl. CPC C07C 45/50; B01J 31/2404

(57) ABSTRACT

The present invention relates to a process for the hydroformylation of 1-olefins on a rhodium-containing complex catalyst comprising a mixture of phosphorus-containing organic complex ligands in the presence of hydrogen and carbon monoxide, wherein the reaction is carried out in a solvent selected from the group of solvents having a boiling point of greater than or equal to 180° C. and less than or equal to 250° C., with a rhodium concentration of greater than or equal to 50 ppm and less than or equal to 250 ppm on catalyst complexes with at least two different complex ligands selected from the group consisting of arylphosphines and cycloalkylphosphines. Furthermore, the present invention relates to the use of the process according to the invention in the context of a two-stage hydroformylation reaction cascade.

The present invention relates to a process for the hydroformylation of 1-olefins on a rhodium-containing complex catalyst comprising a mixture of phosphorus-containing organic complex ligands in the presence of hydrogen and carbon monoxide, wherein the reaction is carried out in a solvent selected from the group of solvents having a boiling point of greater than or equal to 180° C. and less than or equal to 250° C., with a rhodium concentration of greater than or equal to 50 ppm and less than or equal to 250 ppm in the presence of catalyst complexes with at least two different complex ligands selected from the group consisting of arylphosphines and di- or tri-cycloalkylphosphines. Furthermore, the present invention relates to the use of the process according to the invention in the context of a two-stage hydroformylation reaction cascade.

In hydroformylation reactions, olefins are converted to aldehydes with an additional carbon compared to the olefin by means of a synthesis gas mixture of carbon monoxide and hydrogen in the presence of a metal complexed with organic ligands. This reaction principle was developed in the last century by Otto Roelen in Germany and is a fundamental reaction in the field of homogeneous catalysis. The aldehydes obtained can be oxidized to carboxylic acids or hydrogenated to alcohols or converted in other ways in further processing steps. The aldehydes themselves and the other reaction products form important industrial starting materials and are used on a large scale as solvents, additives, raw materials for plasticizers and lubricants, for example.

The hydroformylation process is inherently unspecific with regard to regioselectivity and yields a mixture of linear (n-) and branched (iso)-product aldehydes for 1- or alpha-olefins. Due to the lack of industrially suitable alternative synthesis routes for isoselective reactions, the principle of obtaining a mixture of isomers in this large-scale reaction was accepted. This compromise may be due to the fact that, from a chemical point of view, stereoselective hydroformylation at the C2 carbon position is challenging, since unsubstituted linear 1-olefins have no electronic or steric preferential features. The isomer ratio obtained is a complex function of the prevailing reaction conditions, with the catalyst used, and in particular the formation of the ligand sphere of the catalyst, being considered to have a major influence on the isomeric product composition. In recent years, the majority of industrial interest has focused on process optimization to increase the yield of n-aldehydes. Only recently there has been an increased demand for the corresponding branched aldehydes, whereby in addition to pure stereoselectivity, the economic efficiency of the entire conversion in terms of high selectivities and sufficient conversions must of course also be taken into account as boundary conditions.

The patent literature also contains a large number of process instructions for hydroformylation reactions that are intended to have a special influence on the isomer ratio.

For example, WO 2013 181 188 A1 discloses a process for producing aldehydes comprising: (a) contacting a catalyst composition and a first olefin under hydroformylation conditions to produce a catalyst ligand composition; and b) contacting a second olefin, hydrogen and carbon monoxide in the presence of the catalyst ligand composition to produce aldehydes, wherein the second olefin is propylene, wherein the first olefin has a longer carbon chain than the second olefin, and wherein the catalyst ligand composition comprises tris(3-pyridyl)phosphine, a magnesium-centered tetraphenylporphyrin coordination complex, and a ligand formed in situ by insertion of the first olefin into a rhodium carbonyl bond.

In another patent document, EP 3 156 127 A1, catalyst compositions are described. The catalyst compositions comprise: specific monodentate phosphite ligands; specific monodentate phosphine ligands; and a transition metal catalyst represented by the following Formula 3:

M(L¹)ₓ(L²)_{y}(L³)_{z}

wherein the total content of the entire ligand including the monodentate phosphite ligand and the monodentate phosphine ligand is 1 to 33 moles based on 1 mole of the transition metal catalyst, wherein the ligands of the catalyst R1, R2, R3, R′1, R′2 and R′3 each independently represent a substituted or unsubstituted cycloalkyl or cycloalkenyl group having 5 to 20 carbon atoms; or a substituted or unsubstituted aryl group having 6 to 36 carbon atoms; and when R1, R2, R3, R′1, R′2 and R′ 3 are substituted by a substituent, the substituent is nitro (—NO₂), fluoro (—F), chloro (—Cl), bromo (—Br) or an alkyl group having 1 to 20 carbon atoms; wherein M is selected from the group consisting of cobalt (Co), rhodium (Rh), iridium (Ir), ruthenium (Ru), iron (Fe), nickel (Ni), palladium (Pd), platinum (Pt) and osmium (Os); L¹, L² and L³ are each independently one selected from the group consisting of hydrogen, carbonyl (CO), cyclooctadiene, norbornene, chlorine, triphenylphosphine (TPP) and acetylacetonato (AcAc), and x, y and z are each independently 0 to 5, with the proviso that not all x, y and z are zero, wherein the content of each of the monodentate phosphite ligand and the monodentate phosphine ligand is 0.5 to 32.5 mol based on 1 mol of the transition metal catalyst, wherein a mixing ratio of the monodentate phosphite ligand and the monodentate phosphine ligand is 5:1 to 1:5, on a weight basis.

WO 2009/035204 A1 describes a catalyst composition comprising a triphenylphosphine ligand, a monodentate phosphine ligand, a monodentate phosphine oxide ligand and a transition metal catalyst, and a hydroformylation process using the same. In the hydroformylation process using the catalyst composition according to the present invention, the high catalytic activity can be obtained and the selectivity (n/iso selectivity) with respect to normal or isoaldehyde can be desirably controlled.

Such solutions known from the state of the art can offer further potential for improvement. This relates in particular to the control of the desired isomer ratio while maintaining the boundary condition of a high conversion and a high yield.

It is therefore the task of the present invention to at least partially overcome the disadvantages known from the prior art. In particular, it is the task of the present invention to provide a process which enables control of the isomer ratio, and in particular the presentation of a high proportion of iso-aldehydes, with high conversions and yields. Furthermore, it is the task of the present invention to provide an efficient use of the process, whereby the coupling of the process according to the invention to an upstream process step enables an improved overall process control for the flexible production of different amounts of aldehyde isomers.

The problem is solved by the features of the independent claims, directed to the process according to the invention and the use of the process according to the invention in the context of a multi-step production. Preferred embodiments of the invention are indicated in the dependent claims, in the description or in the figures, whereby further features described or shown in the dependent claims, in the description or in the figures may constitute an object of the invention individually or in any combination, as long as the context does not clearly indicate the contrary.

According to the invention, the problem is solved by a process for the hydroformylation of 1-olefins by reacting 1-olefins on a rhodium-containing complex catalyst comprising a mixture of phosphorus-containing organic complex ligands in the presence of hydrogen and carbon monoxide, the reaction being carried out in a pressure range of greater than or equal to 0.5 MPa and less than or equal to 5 MPa and in a solvent selected from the group of solvents having a boiling point of greater than or equal to 180° C. and less than or equal to 250° C., with a rhodium concentration of greater than or equal to 50 ppm and less than or equal to 250 ppm on catalyst complexes with at least two different complex ligands selected from the group consisting of arylphosphines and di- or tri-cycloalkylphosphines, wherein the proportion of cycloalkylphosphines in the total organophosphorus ligand amount is greater than or equal to 1 mol % and less than or equal to 67 mol % and the molar organophosphorus ligand to rhodium ratio, expressed as molar amount of organophosphorus ligand divided by molar amount of rhodium, is less than or equal to 85.

Surprisingly, it has been found that particularly large quantities of branched aldehydes can be produced from 1-olefins by means of the process control according to the invention in comparison to the process parameters usually available in the prior art, whereby the high proportion of iso-aldehydes is not achieved by a reduction in the olefin conversion or by a reduced selectivity in the direction of the aldehydes as a whole. The reaction can therefore be run advantageously at high rates, high conversions and with very low quantities of undesirable by-products, which naturally influences the economic efficiency of the entire process. Without being bound by theory, this seems in particular to be due to an advantageous combination of the specific ligand environment of the catalyst, the total amount of catalyst used, in combination with the choice of solvent, which on the one hand allows unhindered access of the synthesis gas and on the other hand, due to the steric design of the catalyst environment, leads to high amounts of iso-aldehydes without loss of productivity. Although the fundamental effect of individual ligands on the isomer ratio is known, the shift to the iso-isomer was achieved at the cost of significant losses in productivity. So far, it has not yet been possible to create process conditions that are suitable for efficient production with simultaneous production of high iso-ratios. Furthermore, the process according to the invention is advantageous because the improved process control can be achieved in low pressure ranges and with relatively low catalyst quantities, which also contributes to the improved economic efficiency of the entire process in terms of investment and running costs.

The process according to the invention is a process for the hydroformylation of 1-olefins. 1- or alpha-olefins are substituted or unsubstituted aliphatic or aromatic hydrocarbons which have at least one terminal double bond in the 1-position of the hydrocarbon. The double bond is not incorporated into an aromatic system. Possible carbon numbers of the 1-olefins can, for example, be up to 15, preferably up to 10, more preferably up to 8. Mixtures of different 1-olefins can also be reacted, whereby each of the olefins then has a terminal double bond. Possible representatives of this group can be, for example, ethene, propene, 1-butene, 1-pentene, 1-hexene, 1-octene or styrene. The corresponding olefin reactants can carry further functional groups at other points in the olefin skeleton, provided that these do not prevent the hydroformylation according to the invention from taking place.

The hydroformylation is carried out by reacting the 1-olefins on a rhodium-containing complex catalyst comprising a mixture of phosphorus-containing organic complex ligands in the presence of hydrogen and carbon monoxide. The olefins are hydroformylated, i.e. they are reacted in a reaction zone on a catalyst in the presence of synthesis gas consisting essentially of hydrogen and carbon monoxide, converting the olefinic group into an aldehyde group. The aldehyde obtained has one more carbon atom than the feed olefin. The process according to the invention can be carried out in any suitable reaction vessel. Suitable reaction vessels include, for example, gas sparged reactors, reactors with a liquid overflow, tank reactors with an agitator, so-called trickle-bed reactors, etc. The quantities of synthesis gas supplied and the composition of the same can fluctuate within wide ranges. Typically, the ratios of hydrogen to carbon monoxide can range from 0.5:1 to 10:1, more preferably from 1:1 to 6:1.

The reaction of the olefins to the aldehydes takes place by means of rhodium-metal catalysis, whereby the rhodium is not present as such, but complexed with organic ligands as well as carbon monoxide and hydrogen and thus represents the catalytically active center. The exact composition of the complex, and in particular the stoichiometry of the ligands including the synthesis gas components, is a function of the prevailing reaction conditions. To produce the active catalyst, a rhodium salt is usually introduced into the reaction zone, where it undergoes conversion into the actual active catalyst complex. However, it is also possible for the catalyst to be preformed, i.e. converted into the active species, under similar reaction conditions at a different location outside the reaction zone. Non preformed rhodium components can, for instance, be selected from the group consisting of rhodium(I)dicarbonylacetonyl acetonate, rhodium(II)2-ethylhexanoate, rhodium(II)acetate, rhodium(0)carbonyle (.e.g. Rh₆(CO)I₆, Rh₄(CO)I₂), HRh(CO)(Ph₃P)₃, where Ph is a phenyl group. Mixtures of two or more of these rhodium salts can also be used. It has been found that rhodium 2-ethylhexanoate can preferably be used.

The active catalyst complex always comprises phosphorus-containing organic complex ligands in its coordination sphere in the reaction zone. Phosphorus-containing complex ligands are hydrocarbons, preferably having cyclic groups which have at least one phosphorus atom in the entire hydrocarbon skeleton, whereby the phosphorus atom need not be incorporated into one of the cyclic groups. The phosphorus-containing organic complex ligands can, for example, correspond to the following formula:

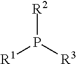

where R¹, R² and R³ are each independently selected from the group consisting of substituted or unsubstituted alkyl groups having 1 to 20 carbon atoms; substituted or unsubstituted cycloalkyl groups or cycloalkenyl groups having 5 to 20 carbon atoms; substituted or unsubstituted aryl groups having 6 to 36 carbon atoms; substituted or unsubstituted heteroalkyl groups with 1 to 20 C atoms; substituted or unsubstituted heteroaryl groups with 4 to 36 C atoms, whereby in the case of substitution of one of the groups, this substitution can have one or more atoms from the group consisting of N, O and S. Possible representatives of these groups are, for example, the triorganophosphines such as triarylphosphine, trialkylphosphine, dialkylarylphosphine, dicycloalkylarylphosphine and tricycloalkylphosphine.

The reaction is carried out in a pressure range of greater than or equal to 0.5 MPa and less than or equal to 5 MPa. Pressures in the specified range can contribute to economically attractive reaction rates with increased iso-selectivities, whereby the costs for the reactor plant are relatively moderate at these relatively low reaction pressures, since the reactor must be designed accordingly less elaborately and the need for additional compressor capacities is reduced.

The reaction takes place in a solvent selected from the group of solvents with a boiling point of greater than or equal to 180° C. and less than or equal to 250° C. The conversion of the olefins to the aldehydes takes place within an inert solvent which dissolves the above-mentioned rhodium complex catalysts as solvent during the reaction. The boiling point of the solvent under standard pressure being in the range indicated above. Possible solvents can be selected from the group of alcohols, acetals or alkanes with a chain length greater than or equal to C8 or mixtures thereof. Possible solvents which fulfill the boiling point criterion according to the invention can, for example, be selected from the group consisting of 2-ethylhexanol, 1-octanol, 1-decanol, higher aldehyde condensation products of one or this specific hydroformylation or mixtures of at least two components from this list. The higher aldehyde condensation products of a hydroformylation are understood to be the bottom product of a hydroformylation, which is formed in the reactor during the course of the reaction. These higher condensation products contain a complex mixture of different components and are also known as thick oils. The aldehyde products are themselves reactive and slowly undergo condensation reactions. This reaction takes place even in the absence of catalysts and is caused by the process control. The liquid condensation products naturally have higher boiling points than the reactant aldehydes. The condensation products can be formed by aldol condensation, for example. Other reaction pathways include Tischshenko reactions, transesterification and dismutation reactions. The condensation products are oligomers of the aldehydes and can also carry other functional groups such as alcohol or ester groups.

The hydroformylation takes place with a rhodium concentration of greater than or equal to 50 ppm and less than or equal to 250 ppm. The concentration of rhodium present in the reaction zone is expressed by the weight ratio of rhodium to the total weight of the solution in the reaction zone. This concentration is based on the ratio of the pure metal weight (without ligands) to the total weight of the solution, including any other components such as dissolved ligands, etc. Lower concentrations can be disadvantageous, as the reaction rate is then too low. Higher concentrations can lead to a reduction in the proportion of iso-aldehyde in the product and, in relation to the costs of the catalyst input, to only disproportionately low increases in the reaction rate.

The catalyst complexes comprise at least two different complex ligands selected from the group consisting of arylphosphines and di- or tri-cycloalkylphosphines. The composition of the ligands has proven to be particularly important for controlling the n/iso aldehyde ratio while maintaining the highest possible reaction rate. High conversions and selectivities can be achieved in particular under certain mixing ratios of arylphosphines and di- or tri-cycloalkylphosphines. Arylphosphines are, for example, compounds of the following formula:

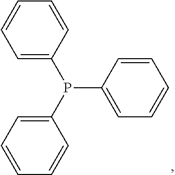 [SMILES: c1ccc(P(c2ccccc2)c2ccccc2)cc1]

whereby the individual aryl groups can still be substituted independently of one another. The group of di- or tri-cycloalkyl-phosphines includes, for example, the following C6-cycloalkyl compounds:

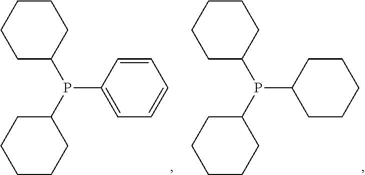 [SMILES: c1ccc(P(C2CCCCC2)C2CCCCC2)cc1 ; C1CCC(P(C2CCCCC2)C2CCCCC2)CC1]

wherein the individual cycloalkyl and/or aryl groups may each independently carry further functional groups as specified above. The cycloalkyl groups can be, for example, C3-C8 cycloalkanes, preferably C4-C7 cycloalkanes.

The proportion of cycloalkylphosphines in the total organophosphorus ligand amount is greater than or equal to 1 mol % and less than or equal to 67 mol %. This narrow range of the cycloalkylphosphine ratio has proved to be particularly suitable for controlling the improved isomer ratio in accordance with the invention while maintaining a high conversion rate. The molar proportion of cycloalkylphosphine is obtained as the quotient of the molar amount of cycloalkylphosphine divided by the total amount of organophosphorus compounds, for example the sum of the compounds with the above formulae from the group of aryl and cycloalkylphosphines. The amount of cycloalkylphosphine can be quantitatively determined using ³¹P methods, for example. The ligands can be introduced purely in the reaction zone or can also be introduced into the reaction solution by adding a preformed metal complex. For clarification, the amount of non-organophosphorus ligands of the rhodium in the reaction zone, for example introduced by the catalyst salt components acetate, ethylhexanoate, CO etc., are not included in the calculation of the molar proportion of cycloalkylphosphines.

The molar ligand to rhodium ratio, expressed as the molar amount of organophosphorus ligand divided by the molar amount of rhodium, is less than or equal to 85. Despite the reduced thermal stability of cycloalkylphosphines, it has proven successful to operate with only a relatively small excess of organophosphorus ligand in relation to the molar amount of rhodium. This ligand content is able to provide the required n/iso ratio, leads to high conversions and is, surprisingly, stable over long production periods in the specified solvents.

In a preferred embodiment of the process, the hydroformylation can be carried out in a temperature range of greater than or equal to 80° C. and less than or equal to 140° C. Sufficient reaction rates can be provided within this temperature range of the reaction zone, whereby in particular a preferred isomer ratio is also obtained at these conversions, which is characterized by a higher proportion of iso-isomers compared to the usual prior art processes.

Within a further preferred embodiment of the process, the molar ratio of arylphosphine to cycloalkylphosphine ligands, expressed as molar amount of arylphosphine divided by molar amount of cycloalkylphosphine ligands, can be greater than or equal to 0.5 and less than or equal to 75. This ratio between arylphosphine and cycloalkylphosphine ligands can provide a significantly increased iso-isomer ratio with only a very small reduction in conversion. In a further preferred embodiment, the ratio may be greater than or equal to 15 and less than or equal to 70, further preferably greater than or equal to 20 and less than or equal to 60.

Within a further preferred aspect of the process, the molar ratio of the arylphosphine ligands to rhodium may be greater than or equal to 5 and less than or equal to 75. In addition to the isomer ratio, the amount of aryl phosphine ligands can also have a significant influence on the productivity of the overall reaction. Within this molar ratio, sufficiently high amounts of iso-isomers can be provided with high conversions. The molar ratio can furthermore preferably be greater than or equal to 35 and less than or equal to 65, furthermore preferably greater than or equal to 45 and less than or equal to 55.

In a further preferred embodiment of the process, the molar ratio of the cycloalkylphosphine ligands to rhodium may be greater than or equal to 1 and less than or equal to 10. The amount of cycloalkylphosphine ligands can in particular exert a significant influence on the isomer ratio and in particular on the iso proportion of the aldehydes formed. At lower ratios, the influence of the cycloalkylphosphine ligands on the increase in the iso-aldehyde content is too small. Higher ratios can be disadvantageous, since in these cases the olefin conversion rates can be significantly reduced. The molar ratio may further preferably be greater than or equal to 2 and less than or equal to 8, further preferably greater than or equal to 4 and less than or equal to 6.

Within a preferred aspect of the process, the arylphosphine can be triphenylphosphine. The use of triarylphosphine (TPP) as arylphosphine can contribute to particularly high yields and particularly long service lives of the catalyst solution. TPP in excess is particularly capable of stabilizing the rhodium in the solution. In addition, TPP can also act as a ligand reservoir in the event of damage to the cycloalkylphosphine ligand.

Within a preferred aspect of the process, the cycloalkylphosphine can be tricyclohexylphosphine. In particular, the use of tricyclohexylphosphine can contribute to a particularly efficient shift of the aldehyde isomer ratio towards the iso-isomers. This is most likely achieved by the increased space requirement of the ligand. The shift of the isomer ratio towards iso-aldehydes occurs in the specified solvents even at concentrations that do not yet have a detrimental effect on the possible achievable reaction rate. In this respect, this ligand can contribute more efficiently than the cycloalkylphosphines with only two cycloalkyl groups.

In a further preferred embodiment of the process, the 1-olefin can be selected from the group consisting of C3-C8 olefins or mixtures thereof. The middle olefins in particular can be increasingly converted towards the iso-aldehyde isomers using the process according to the invention without major losses in conversion. Without being bound by theory, this effect with the “middle” alpha-olefins results from the special orientation of the olefin on the catalyst complex, which is determined by the ligand composition and the solvent.

In a preferred embodiment of the process, the molar ratio of synthesis gas to 1-olefin, expressed as (molar amount of H₂+molar amount of CO) divided by molar amount of 1-olefin, can be greater than or equal to 1:1 and less than or equal to 5:1. Within this ratio between olefin and synthesis gas, sufficiently high concentrations of reactants can be provided in the claimed group of solvents, which together lead to high conversions and only a small number of undesired side reactions.

Further according to the invention is the use of the process according to the invention for the hydroformylation of 1-olefins on a complex catalyst, wherein the hydroformylation is carried out in two steps, wherein within a first process step the reaction is carried out in the presence of a rhodium-containing complex catalyst comprising arylphosphine ligands and without cycloalkylphosphine ligands in a solvent selected from the group of alcohols, acetals or alkanes with a chain length greater than or equal to C10 or a mixture thereof, and wherein within a second process step a further solvent with a boiling point of greater than or equal to 180° C. and less than or equal to 250° C. and additionally cycloalkylphosphine ligands are added to the reaction mixture of the first process step.

Surprisingly, it has been found that the process according to the invention can be used very advantageously as part of a two-step hydroformylation cascade. In this now two-step process, several advantages can be achieved by setting the reaction conditions according to the invention only in the second step. By performing the second step, the reaction solution in the reaction zone of the first step can essentially also be used, whereby the adjustment to the conditions according to the invention can only be achieved by adding ligands and solvent. Complex separation operations or even an exchange of the entire reaction solution can be omitted. By coupling the two process steps, a desired isomer ratio can also be set via both process steps, whereby the position of the ratio is also determined by the setting and the position of the second step. In addition, the thick oils formed in the first step can advantageously also be used and part of the solvent addition with very high boiling points can be omitted, as these are already present, at least in part, in the reaction zone.

In the use according to the invention, the hydroformylation is carried out in two steps, wherein within a first process step the reaction is carried out in the presence of a rhodium-containing complex catalyst comprising arylphosphine ligands and without cycloalkylphosphine ligands in a solvent selected from the group of alcohols, acetals or alkanes with a chain length greater than or equal to C10 or a mixture thereof. The first process step of the cascade is therefore not carried out according to the invention with the exclusion of cycloalkyl ligands. In this process step, more n-aldehydes are formed. Naturally, thick oils are also formed during this step, which originate from the inherent condensation of the aldehydes produced. The process conditions within this step may not correspond to those of the process according to the invention, i.e. the rhodium concentrations in the reaction zone, for example, may be higher than claimed in the process according to the invention.

Within a second process step, a further solvent with a boiling point of greater than or equal to 180° C. and less than or equal to 250° C. and additionally cycloalkylphosphine ligands are added to the reaction mixture of the first process step. By adding the further solvent with a high boiling point, the reaction solution of the first process step is adjusted to the composition of the process according to the invention. The dilution and the addition of the further ligand species now produce reaction conditions which generate a higher proportion of iso-isomers without significant losses in conversion and productivity. This is advantageously possible and leads to an efficient use of the reaction solution by using part of the reaction environment of the first step. In addition, depending on the isomer requirement, the proportion of available isomers can advantageously be controlled via the lengths of the individual process steps. The result is a synergistically interacting overall process.

Within a further preferred aspect of the use, the solvent in the second process step may be selected from the group consisting of alcohols having a chain length greater than or equal to C8, acetals having a chain length greater than or equal to C13, alkanes having a chain length greater than or equal to C10, or mixtures of at least two components of this group. This group of additionally used solvents in the second process step can contribute to the fact that improved catalyst service lives can be obtained at high conversion rates, in particular for the second process step.

According to a preferred characteristic of the use, the weight ratio of the solvent added in the second process step to the solvent quantity of the first process step can be greater than or equal to 4 and less than or equal to 20. To obtain the most efficient solvent mixture possible, consisting of the high-boiling solvent formed in the first process step and the high-boiling solvent added in the second process step, the above mixing ratio of the two solvents has proved to be particularly suitable. In the second process step, long catalyst lifetimes, high conversions and a high proportion of iso-isomers for the aldehydes are obtained.

In a further preferred embodiment of the use, the concentration of the arylphosphine ligands in the first process step can be greater than or equal to 10% by weight and less than or equal to 30% by weight, based on the total weight of the process solution. This concentration of aryl phosphine ligands in the first process step has proven to be particularly suitable for particularly efficient adjustment of the ligand ratios according to the invention in the second process step. This concentration leads to a stable conversion with relatively low catalyst deactivation in the first process step, but at the same time is also low enough to avoid wasting too much of the catalyst to be added in the second process step. An efficient overall process is obtained across both process steps, which, in addition to suitable control of the n/iso ratios, also shows high overall conversion rates.

Within a preferred aspect of the use, the concentration of cycloalkylphosphines added in the second process step can be greater than or equal to 0.01% by weight and less than or equal to 1% by weight based on the total weight of the process solution. For process economy and for an efficient increase in the iso content in the product, it has been found that only relatively small amounts of cycloalkylphosphine need to be added to the reaction solution of the second process step. The addition of cycloalkylphosphine in particular increases the formation of iso-aldehydes, whereby the selected reaction conditions can prevent a sharp drop in conversion in the second process step compared to the first process step.

Within a preferred aspect of the use, the total amount of rhodium can be added in the first process step. For simplified process control, to obtain a preferred high iso-ratio of the aldehydes formed and for high conversions, it has been found to be suitable for the total addition of the metallic complex catalyst to take place in the first process step. This is surprising, since in the second process step, due to the changed ligand supply, an equilibrium must first be established, which, as expected, should happen more quickly with the addition of fresh catalyst due to the changed equilibrium position. Surprisingly, this is not the case.

Further details, features and advantages of the object of the invention are apparent from the dependent claims and from the following description of the figures and the associated examples. It shows the:

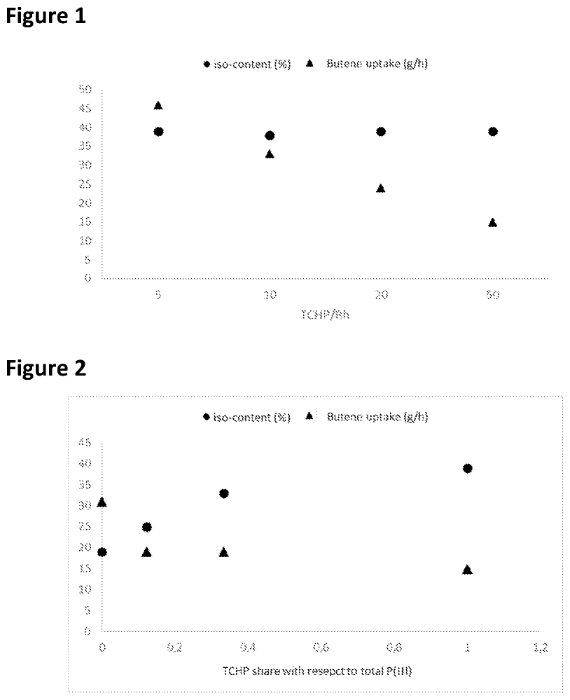

FIG. 1 the iso-aldehyde content and the 1-butene uptake of a reaction solution with 2-ethylhexanol as solvent as a function of the molar tricyclohexylphosphine/rhodium ratio;

FIG. 2 the iso-aldehyde fraction and the 1-butene uptake of a reaction solution with 2-ethylhexanol as solvent as a function of the tricyclohexylphosphine fraction of organophosphorus ligands;

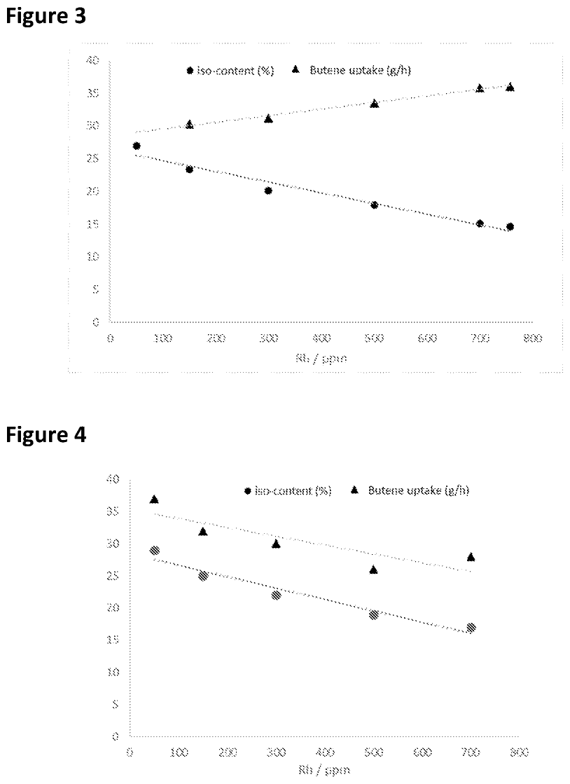

FIG. 3 the iso-aldehyde content and the 1-butene uptake of a reaction solution with Rh-triphenylphosphine catalyst after dilution with 2-ethylhexanol as solvent, in the absence of tricyclohexylphosphine, as a function of the rhodium concentration;

FIG. 4 the iso-aldehyde content and the 1-butene uptake of a reaction solution with Rh-triphenylphosphine catalyst after dilution with 2-ethylhexanol as solvent, and with tricyclohexylphosphine, as a function of the rhodium concentration.

### EXAMPLES

All experiments were carried out in a reactor in batch mode with a preformed catalyst phase (9 bar synthesis gas pressure (SynGas), temperature 120° C. for 30 min). A continuous addition of 1-butene/syngas 1:1 with 13 bar at 120° C. was carried out, whereby the reaction was run with varying reaction times of 20 min to 2 h up to a conversion of 25 g 1-butene. In all experiments, the catalyst solution was transferred to the evacuated reactor under inert conditions and the reaction was started with a continuous addition of 1-butene and synthesis gas. The resulting 1-butene uptake over time (system productivity) and the iso/n ratio of the C5 aldehydes were used as characteristic values to determine the 2-methylbutanal (2-MB) content.

In the first series of experiments, the influence of tricyclohexylphosphine (TCHP) with varying TCHP/Rh ratios on the iso/n ratio of the C5 aldehydes formed and the 1-butene uptake was investigated. The results are shown in Table 1 and FIG. 1:

TABLE 1
Hydroformylation of 1-butene with TCHP in 2-ethylhexanol (2-EHol)

| | | | | | TCHP | Conversion | | |
|---|---|---|---|---|---|---|---|---|
| | | Rh | | | (% by | % according | | 1-Butene |
| Exp. | LM | (ppm) | TPP/Rh | TCHP/Rh | weight) | to GC | iso:n | (g/h) |
| 1. | 2-EHol | 50 | 65 | 0 | 0 | 99 | 24:76 | 43 |
| 2. | 2-EHol | 50 | 0 | 5 | 0.07 | 94 | 39:61 | 46 |
| 3. | 2-EHol | 50 | 0 | 10 | 0.14 | 92 | 38:62 | 33 |
| 4. | 2-EHol | 50 | 0 | 20 | 0.27 | 91 | 39:61 | 24 |
| 5. | 2-EHol | 50 | 0 | 50 | 0.68 | 82 | 39:61 | 15 |

Table 1 and FIG. 1 show that TCHP increases the proportion of 2-MB in the product compared to TPP and thus leads to an increase in the iso/n ratio. Even with a low TCHP/Rh ratio, the ligand has a positive effect on the formation of the branched aldehyde. The high space requirement of the TCHP ligand and the resulting change in the geometry of the Rh complex and its properties probably lead to a change in the selectivity of the 1-butene hydroformylation. Under higher TCHP/Rh ratios, however, the 1-butene uptake and thus the reaction rate of the overall system also decreases. The reduced olefin uptake can probably be attributed to the high binding affinity of the TCHP ligand to rhodium.

In a further series of tests, a ligand mixture of TCHP and triphenylphosphine (TPP) was used for the process according to the invention. If present, a TCHP-Rh ratio of 50 was specified and the amount of TPP in the reaction solution was varied. The other specific experimental conditions are given above. The results are shown in Table 2.

TABLE 2
Hydroformylation of 1-butene with TCHP and TPP in 2-ethylhexanol (2-EHol)

| | | | | | TCHP | Conversion | | |
|---|---|---|---|---|---|---|---|---|
| | | Rh | | | (% by | % according | | 1-Butene |
| Exp. | LM | (ppm) | TPP/Rh | TCHP/Rh | weight) | to GC | iso:n | g/h |
| 1. | 2-EHol | 50 | 65 | 0 | 0 | 99 | 24:76 | 43 |
| 5. | 2-EHol | 50 | 0 | 50 | 0.68 | 82 | 39:61 | 15 |
| 6. | 2-EHol | 50 | 100 | 50 | 0.68 | 76 | 33:67 | 19 |
| 7. | 2-EHol | 50 | 360 | 50 | 0.68 | 79 | 25:75 | 19 |
| 8. | 2-EHol | 50 | 360 | 0 | 0 | 87 | 19:81 | 31 |

FIG. 2 shows the dependence of the obtainable iso proportion and the butene uptake as a function of the TCHP proportion of the total phosphorus(III) introduced by the ligands. Equivalent to the TCHP/Rh ratio, the higher the TCHP content, the higher the iso content in the product, but the butene uptake and thus the reaction rate also decrease significantly. By using TPP and TCHP ligands together, the reaction can be run at high conversion rates and high iso ratios under specific concentration and proportion relationships between ligand and catalyst and between the ligands themselves. The use of TPP leads to higher productivities, which are maintained even at higher TPP/Rh ratios. At the same time, a larger TPP surplus stabilizes the catalyst system against deactivation, so that long process runtimes of the catalyst solution can be achieved. The addition of a small excess of TCHP in relation to the molar amount of rhodium is able to increase the 2-MB content in the reaction solution while maintaining the same catalyst activity.

FIG. 3 shows the iso-aldehyde content and the 1-butene uptake of a reaction solution with Rh-triphenylphosphine catalyst after dilution with 2-ethylhexanol as solvent, in the absence of tricyclohexylphosphine, as a function of the rhodium concentration. The TPP-Rh ratio was 66 in all experiments. The dilution of the catalyst solution with 2-ethylhexanol results in a lower catalyst concentration, which accordingly results in a lower 1-butene conversion. The linear regressions over the two parameters result in an expected linear relationship between butene uptake and the iso-ratio of the catalyst concentration when using a pure TPP ligand system. As the Rh concentration increases but the TPP/Rh ratio remains constant, the productivity of the catalyst system increases, whereas the iso content in the product decreases when a pure TPP ligand system is used. In order to counteract the decreasing iso content and thus increase the formation of 2-MB, TCHP ligand is added to the Rh-TPP catalyst system in the process according to the invention.

FIG. 4 shows the iso-aldehyde content and the 1-butene uptake of a reaction solution with mixed Rh-TPP-TCHP catalyst in a dilution series with 2-ethylhexanol as solvent as a function of the rhodium concentration. The experiment thus essentially corresponds to the procedure described for FIG. 4, except that a mixed ligand system of TPP and TCHP is used in this series. Surprisingly, when a ligand mixture (TPP/Rh 69 and TCHP/Rh 2) is used, there is both an increase in butene uptake and an increase in the iso-aldehyde content as the catalyst metal concentration decreases with dilution. Thus, surprisingly, the curve is fundamentally different from the curve for a pure TPP catalyst system, as discussed with reference to FIG. 4. This correlation clearly shows that by using a ligand system according to the invention comprising two ligands (TPP/TCHP) in a solvent, with relatively low catalyst concentrations and a specific quantitative ratio of the two ligands to each other, surprisingly high conversions as well as high iso-aldehyde contents can be realized. The particular advantages of the process according to the invention are based on the specific quantitative ratio of the two ligands. On the one hand, only small molar quantities of TCHP ligand in relation to the rhodium metal are sufficient to increase the iso content in the product due to the spatially demanding ligand. The catalyst activity is also very high with this ligand and low TCHP/Rh ratio. On the other hand, the molar amount of TPP ligand relative to the rhodium metal was selected such that the rhodium-ligand catalyst system is stabilized and the catalyst activity is maintained.

1. A process for the hydroformylation of 1-olefins by reacting 1-olefins on a rhodium-containing complex catalyst comprising a mixture of phosphorus-containing organic complex ligands in the presence of hydrogen and carbon monoxide, characterized in that the reaction is carried out in a pressure range of greater than or equal to 0.5 MPa and less than or equal to 5 MPa, and in a solvent selected from the group of solvents having a boiling point of greater than or equal to 180° C. and less than or equal to 250° C., with a rhodium concentration of greater than or equal to 50 ppm and less than or equal to 250 ppm on catalyst complexes with at least two different complex ligands selected from the group consisting of arylphosphines and di- or tri-cycloalkylphosphines, wherein the proportion of cycloalkylphosphines in the total organophosphorus ligand amount is greater than or equal to 1 mol % and less than or equal to 67 mol % and the molar organophosphorus ligand to rhodium ratio, expressed as molar amount of organophosphorus ligand divided by molar amount of rhodium, is less than or equal to 85.

2. The process according to claim 1, wherein the hydroformylation is carried out in a temperature range of greater than or equal to 80° C. and less than or equal to 140°° C.

3. The process according to claim 1, wherein the molar ratio of arylphosphine to cycloalkylphosphine ligands, expressed as molar amount of arylphosphine divided by molar amount of cycloalkylphosphine ligands, is greater than or equal to 0.5 and less than or equal to 75.

4. The process according to claim 1, wherein the molar ratio of the arylphosphine ligands to rhodium is greater than or equal to 5 and less than or equal to 75.

5. The process according to claim 1, wherein the molar ratio of cycloalkylphosphine ligands to rhodium is greater than or equal to 1 and less than or equal to 10.

6. The process according to claim 1, wherein the arylphosphine is triphenylphosphine.

7. The process according to claim 1, wherein the cycloalkylphosphine is tricyclohexylphosphine.

8. The process according to claim 1, wherein the 1-olefin is selected from the group consisting of C3-C8 olefins or mixtures thereof.

9. The process according to claim 1, wherein the molar ratio of synthesis gas to 1-olefin, expressed as (molar amount of H₂+molar amount of CO) divided by molar amount of 1-olefin, is greater than or equal to 1:1 and less than or equal to 5:1.

10. Use of the process according to claim 1, for the hydroformylation of 1-olefins on a complex catalyst, characterized in that the hydroformylation is carried out in two steps, wherein within a first process step the reaction is carried out in the presence of a rhodium-containing complex catalyst comprising arylphosphine ligands and without cycloalkylphosphine ligands in a solvent selected from the group consisting of alcohols, acetals or alkanes with a chain length greater than or equal to C10 or a mixture thereof, and wherein within a second process step a further solvent with a boiling point greater than or equal to 180° C. and less than or equal to 250° C. and additionally cycloalkylphosphine ligands are added to the reaction mixture of the first process step.

11. Use according to claim 10, wherein the solvent in the second process step is selected from the group consisting of alcohols having a chain length greater than or equal to C8, acetals having a chain length greater than or equal to C13, alkanes having a chain length greater than or equal to C10, or mixtures of at least two components of this group.

12. Use according to claim 10, wherein the weight ratio of the solvent added in the second process step to the amount of solvent of the first process step is greater than or equal to 4 and less than or equal to 20.

13. Use according to claim 10, wherein the concentration of the arylphosphine ligands in the first process step is greater than or equal to 10% by weight and less than or equal to 30% by weight based on the total weight of the process solution.

14. Use according to claim 10, wherein the concentration of the cycloalkylphosphine added in the second process step is greater than or equal to 0.01% by weight and less than or equal to 1% by weight based on the total weight of the process solution.

15. Use according to claim 10, wherein the total amount of rhodium is added in the first process step.
